(19) United States
(12) Patent Application Publication
(10) Pub. No.: US 2016/0022714 A1
(43) Pub. Date: Jan. 28, 2016

(54) O-GLCNACYLATION TREATMENT FOR ISCHEMIC BRAIN INJURY

(71) Applicant: THE RESEARCH FOUNDATION FOR MENTAL HYGIENE, INC., Menands, NY (US)

(72) Inventor: Cheng-Xin Gong, Staten Island, NY (US)

(73) Assignee: THE RESEARCH FOUNDATION FOR MENTAL HYGIENE, INC., Menands, NY (US)

(21) Appl. No.: 14/808,359

(22) Filed: Jul. 24, 2015

Related U.S. Application Data
(60) Provisional application No. 62/029,812, filed on Jul. 28, 2014.

(51) Int. Cl. A61K 31/7008; A61K 31/437; A61K 9/00; A61K 31/429

(52) U.S. Cl. CPC A61K 31/7008; A61K 31/429; A61K 31/437; A61K 9/0019

(57) ABSTRACT

Cerebral ischemia-reperfusion injury may be treated by compounds that increase brain O-GlcNAcylation, such as a therapeutic amount of a compound that increases the hexosamine biosynthesis pathway flux that bypasses glutamine/fructose-6-phosphate amidotransferase 2 or a therapeutic amount of a compound that inhibits OGA. The initial and transient elevation of brain O-GlcNAcylation is neuroprotective and helps ameliorate cerebral ischemia-reperfusion injury when administered within three hours of the ischemia-reperfusion-induced brain injury and continues for at least two days.

### CROSS-REFERENCE TO RELATED APPLICATIONS

The present application claims the benefit of U.S. Provisional Application No. 62/029,812, filed on Jul. 28, 2014.

### BACKGROUND OF THE INVENTION

1. Field of the Invention

The present invention relates to brain injury treatments and, more specifically, to the use of O-GlcNAcylation to ameliorate ischemia-reperfusion-induced brain injury.

2. Description of the Related Art

Stroke is mostly caused by occlusion of a cerebral artery (ischemic stroke) and is a leading cause of brain injury that strikes approximately 800,000 people and kills approximately 150,000 each year in the United States alone. Ischemia causes severe nutritional stress due to oxygen and glucose deprivation. Several studies have reported alteration of O-GlcNAcylation level upon glucose deprivation in cultured cells.

Protein O-GlcNAcylation is a unique type of posttranslational modification of nucleocytoplasmic proteins with β-N-acetylglucosamine (GlcNAc). Protein O-GlcNAcylation is catalyzed by O-GlcNAc transferase (OGT), and the O-GlcNAc group on proteins can be removed with the catalysis of β-N-acetylglucosaminidase (O-GlcNAcase, OGA). O-GlcNAcylation is regulated dynamically by these two enzymes and by the intracellular concentration of UDP-GlcNAc, a product of glucose metabolism through the hexosamine biosynthetic pathway (HBP). Up to 5% of glucose imported into the cell is metabolized through the HBP.

Protein O-GlcNAcylation plays a critical role in regulating numerous biological processes, such as transcription, translation, protein degradation, signal transduction, and cell survival. O-GlcNAcylation also serves as a nutrient and stress sensor of the cell. A range of stress stimuli cause an acute increase in protein O-GlcNAcylation. During ischemia, cells experience a severe depletion of extracellular glucose and oxygen, resulting in nutritional stress responses.

Marked changes of O-GlcNAcylation are seen in ischemic heart tissue and in diabetes. However, the role of O-GlcNAcylation under these conditions is controversial. O-GlcNAcylation is reported to be protective against heart ischemia-reperfusion injury, but it is shown to produce adverse effects on heart function in diabetic hearts. Inhibition of the acute elevation of O-GlcNAc levels increases cell death, and augmentation of O-GlcNAc increases the tolerance to stress in vitro.

Accordingly, there is a need in the art for a method of treating ischemia-reperfusion-induced brain injury that properly modulates the levels of O-GlcNAcylation in the brain.

### BRIEF SUMMARY OF THE INVENTION

The present invention comprises the modification of O-GlcNAcylation levels to ameliorate ischemia-reperfusion-induced brain injury. More particularly, the invention involves a method of treating a patient for ischemia-reperfusion-induced brain injury by modulating the levels of O-GlcNAcylation in the brain of the patient. The levels of O-GlcNAcylation in the brain of the patient may be modulated by administering a sufficient amount of a therapeutic agent to increase the hexosamine biosynthesis pathway flux that bypasses glutamine/fructose-6-phosphate amidotransferase 2. For example, glucosamine may be administered by injection, such as intravenous injection. Alternatively, the levels of O-GlcNAcylation in the brain of the patient may be modulated by administering a sufficient amount of a therapeutic agent to inhibit OGA, such as oral administration of thiamet-G. For example, a method of treating a patient for ischemia-reperfusion-induced brain injury may comprise the step of modulating the levels of O-GlcNAcylation in the brain of the patient, wherein the step of modulating the levels of O-GlcNAcylation in the brain of the patient comprises administering a therapeutic amount of a compound that increases the hexosamine biosynthesis pathway flux that bypasses glutamine/fructose-6-phosphate amidotransferase 2. For example, the compound may comprise glucosamine that is administered by intravenous injection in a dosage between 1 and 10 mg per day. Preferably, the treatment begins within three hours of the ischemia-reperfusion-induced brain injury and continues for at least two days. The treatment may also comprise the step of modulating the levels of O-GlcNAcylation in the brain of the patient comprises administering a therapeutic amount of a compound that inhibits O-linked N-acetylglucosamine. For example, the compound may comprise thiamet-G administered by intravenous injection in a dosage between 0.1 and 1 mg per day or administered orally in a dosage of between 1 and 10 mg per day. Alternatively, the compound may comprise GlcNAcstatin G administered by intravenous injection in a dosage between 0.02 and 0.2 mcg per day or orally in a dosage between 0.2 and 2 mcg per day.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1A through 1D are a series of images and charts showing dynamic alterations of O-GlcNAcylation in the cerebral cortex after ischemia and ischemia-reperfusion injury, as follows: FIG. 1A are Western blots of global O-GlcNAcylated proteins in the cerebrocortical homogenates from mice 0-12 hrs after MCAO. FIG. 1B is a quantification of the blots shown in panel A. FIG. 1C are Western blots of global O-GlcNAcylated proteins in the cerebrocortical homogenates from mice after MCAO for 2 hrs, followed by reperfusion for the indicated times (0, 3 or 12 hrs). FIG. 1D is a quantification of the blots shown in FIG. 1C. Data are presented as Mean±SEM. *, p<0.05 vs. contralateral; #, p<0.05 vs. 0 h ipsilateral group. S, sham; C, contralateral; I, ipsilateral;

FIG. 2 is a series of images showing the topography of the elevations of O-GlcNAcylation after MCAO. Frozen coronal brain sections of mice sacrificed 1 (A-E) or 2 h (F-L) after MCAO at the right side were stained with a mixture of monoclonal antibodies RL2 and CTD110.6 for O-GlcNAc (green) and with TO-PRO (blue) for nuclear staining;

FIG. 3A through FIG. 3C is a series of images and charts showing the levels of GLUT1, GLUT2, GLUT3, GFAT2, OGT and OGA in the cerebral cortex after ischemia and ischemia-reperfusion injury, where: FIGS. 3A and 3C are Western blots of cerebrocortical homogenates from mice 0-12 hrs after MCAO, FIG. 3A, or from mice after MCAO for 2 hrs, followed by reperfusion for 0-12 hrs, FIG. 3C. FIGS. 3B and 3D are quantifications of the blots (mean±SEM) after normalization with the β-actin level. *, p<0.05 vs. contralateral side. FIG. 3E are graphs of the O-GlcNAcylation levels in the mouse brains plotted against the levels of the upper band of GFAT2 and the linear correlation. S, sham; C, contralateral; I, ipsilateral;

FIG. 4A through 4D are a series of images and charts showing Coomassie blue stating of the cerebral cortical proteins and blood glucose level after ischemia-reperfusion injury, where FIGS. 4A and B are Coomassie blue staining of cerebrocortical homogenate proteins from mice 0-12 hrs after MCAO, FIG. 4A, or from mice after MCAO for 2 hrs, followed by reperfusion for 0-12 hrs, FIG. 4B. FIGS. 4C and 4D are graphs of Blood glucose concentration (mean±SEM) of mice 0-12 hrs after MCAO, FIG. 4C, or mice after MCAO for 2 hrs, followed by reperfusion for 0-12 hrs, FIG. 4D. *, p<0.05 vs. sham group;

FIGS. 5A through D are a series of images showing the levels of global O-GlcNAcylation, phosphoserine (P-Ser), phosphothroenine (P-Thr), and phosphorylation levels GSK-3β and CaMK-II. Western blots of cerebrocortical homogenates from mice after MCAO for 2 hrs, followed by reperfusion for 0-12 hrs, are shown;

FIGS. 6A through 6I are a series of images, schematics and charts showing the modulation of ischemia-reperfusion injury by O-GlcNAcylation, where: FIG. 6A is a diagram of HBP and O-GlcNAcylation pathway. The pharmacological manipulations used in this study are indicated in blue color. FIG. 6B is a schematic presentation of the experimental design. FIG. 6C are Western blots for O-GlcNAcylation of striatum homogenates from mice 2 hrs after MCAO. FIG. 6D is a quantification of the blots shown in panel C. *, p<0.05 vs. contralateral; #, p<0.05 vs. saline group. FIG. 6E is TTC staining of the brain slices from mice after MCAO for 2 hrs followed by reperfusion for 24 hrs. FIG. 6F is a Quantification of the infarct sizes detected by TTC staining *, p<0.05 vs. saline group. FIG. 6G are scores of motor deficits detected after MCAO for 2 hrs and reperfusion for 1 hr. The numbers in the columns indicate the number of mice. *, p<0.05 vs. saline group. FIGS. 6H and 6I are Western blots for O-GlcNAcylation of striatum homogenates from mice 2 hrs after MCAO. The mice received icy injection of the indicated amounts of thiamet-G 24 hrs before MCAO. FIG. 6J is TTC staining of the brain slices from mice after MCAO for 2 hrs followed by reperfusion for 24 hrs. FIG. 6K is a quantification of the infarct sizes detected by TTC staining *, p<0.05 vs. saline group. FIG. 6L are scores of motor deficits detected after MCAO for 2 hrs and reperfusion for 1 hr. The numbers indicate the number of mice per group;

FIGS. 7A through 7G are a series of images, schematics and charts showing infarct size, motor deficit and mortality after ischemia-reperfusion injury in neuronal OGT KO mice, where FIG. 7A is schematic presentation of the experimental design. FIG. 7B is Western blots for the levels of OGT and O-GlcNAcylation in the striatum of mice 2 hrs after MCAO. FIG. 7C is a quantification of the blots shown in panel B. *, p<0.05 vs. control group; #, p<0.05 vs. contralateral group. FIG. 7D is TTC staining of the brain slices from mice after MCAO for 2 hrs followed by reperfusion for 24 hrs. FIG. 7E is a quantification of the infarct sizes detected by TTC staining FIG. 7F are scores of motor deficits. The numbers in the columns indicate the number of mice. FIG. 7G is a chart of mortality of mice within 24 hrs after MCAO/reperfusion;

FIGS. 8A through 8Q are a series of images showing the histology and O-GlcNAc immunohistochemistry of ischemic human brain tissue. Adjacent paraffin sections of the middle frontal gyms of the postmortem brain from an 82 year old female individual were stained with hematoxylin and eosin (H&E), FIGS. 8A through 8H, or monoclonal antibody RL2 against O-GlcNAcylated proteins, FIGS. 8I through 8P. Control staining was carried out when RL2 was eliminated from the primary antibody solution, FIG. 8Q. Asterisks in panels A and I indicate the ischemic area. White rectangles and squares indicate the areas for images of higher magnifications. # indicates the tissue marker used for identification of the same areas in the adjacent sections. Arrowheads in panel H indicate macrophages.

### DETAILED DESCRIPTION OF THE INVENTION

The present invention comprises the use of O-GlcNAcylation to ameliorate ischemia-reperfusion-induced brain injury. The MCAO mouse model was used to find, for the first time, a dynamic alteration of brain protein O-GlcNAcylation during cerebral ischemia and during ischemia-reperfusion injury. An initial marked elevation (1-4 hours after ischemia) and then marked decline of protein O-GlcNAcylation was demonstrated during permanent cerebral ischemia. If reperfusion occurred two hours after ischemia, the elevation of O-GlcNAcylation lasted longer (up to at least 12 hrs after reperfusion, the longest time point). Most importantly, the transient elevation of brain O-GlcNAcylation helps ameliorate cerebral ischemia-reperfusion injury.

O-GlcNAcylation is a known sensor of intracellular glucose metabolism, and decreased intracellular glucose usually results in decreased O-GlcNAcylation because the donor for O-GlcNAcylation, UDP-GlcNAc, is generated from glucose metabolism through HBP. However, protein O-GlcNAcylation also responds to various stresses. Transient elevation of O-GlcNAcylation is known to occur under conditions of decreased intracellular glucose concentration. A transient elevation of O-GlcNAcylation was reported after glucose deprivation in cultured HepG2 cells, Neuro-2a neuroblastoma cells, and cardiomyocytes. Interestingly, the mechanisms leading to the transient elevation of O-GlcNAcylation appear to be different among various cell types and/or conditions. In Hep2G cells, glucose deprivation stimulates O-GlcNAcylation through up-regulation of OGT and concomitant decrease in OGA levels. In Neuro-2a cells, glucose deprivation caused only up-regulation of OGT expression. In cardiomyocytes, the glucose deprivation-induced increase in O-GlcNAcylation was calcium-dependent. Here, the OGT level was not altered during cerebral ischemia-reperfusion injury and thus the transient elevation of O-GlcNAcylation does not appear to result from any changes in the expression of OGT.

In an effort to elucidate the possible molecular mechanism leading to the dynamic alteration of O-GlcNAcylation during cerebral ischemia-reperfusion injury, brain GLUTs, GFAT2 (the major rate-limiting enzyme of HBS pathway in the brain), OGT and OGA were studied. Changes in the levels of these proteins critical to the regulation of O-GlcNAcylation were observed, but not GLUT2 or OGT. However, except with the appearance of a GFAT2 band with an upward mobility shift in SDS-PAGE (˜77 kDa), the alterations of these proteins did not coincide well with and thus could not explain the dynamic alteration of O-GlcNAcylation during cerebral ischemia-reperfusion injury. In contrast, the appearance of the ˜77 kDa GFAT2 coincided precisely with the transient elevation of O-GlcNAcylation in the ischemic brain tissue. Further experiments suggest that this high molecular weight GFAT2 is most likely the phosphorylated and activated GFAT2. An up-shift of apparent molecular weight in SDS-PAGE is actually a common phenomenon of protein phosphorylation. GFAT2 has been previously reported to be activated upon phosphorylation. The elevation of O-GlcNAcylation during the early phase of cerebral ischemia may be caused by activation of GFAT2, which in turn results in more intracellular concentration of UDP-GlcNAc and consequently elevated O-GlcNAcylation. A strong positive correlation between the level of the ˜77 kDa GFAT2 and the O-GlcNAcylation level further supports this conclusion. The stress-induced hyperglycemia, which also occurred when O-GlcNAcylation was elevated during the early phase of the cerebral ischemia-reperfusion injury, could also partially contribute to the transient elevation of O-GlcNAcylation in the brain through more glucose entering the HBP pathway leading to more UDP-GlcNAc. UDP-GlcNAc is also a strong activator of OGT and can markedly activate OGT activity without changing the level of the enzyme.

How GFAT2 is over-activated during the early phase of cerebral ischemia is appropriate for further investigation. It is well established that ischemia induces abnormal calcium influx and the consequent over-activation of calcium-dependent enzymes including CaMK-II. This kinase has been reported to phosphorylate and activate GFAT. Thus, the GFAT2 activation during the early phase of cerebral ischemia could have resulted from CaMK-II over-activation in the ischemic mouse brains.

It is well documented that O-GlcNAcylation regulates phosphorylation of the same proteins and these two posttranslational modifications sometimes regulate each other in a reciprocal manner. In the ischemic brain tissue, when the global protein O-GlcNAcylation was increased, the global protein phosphorylation at both serine and threonine residues was decreased. Because protein phosphorylation is known to be a critical regulator of diverse aspects of neuronal function, the overall neuroprotective role of the elevation of O-GlcNAcylation during the early phase of cerebral ischemia might act partially through downregulation of the phosphorylation of some critical neuronal proteins.

To explore the possible role of the dynamic elevation of O-GlcNAcylation in cerebral ischemia, three pharmacological compounds were used (DON, glucosamine and thiamet-G) to manipulate the level of O-GlcNAcylation. A moderate elevation and reduction of brain O-GlcNAcylation was observed with glucosamine and DON, respectively. In addition to glucosamine and thiamet-G, a group of compounds called GlcNAcstatins may be used as they are very potent and selective OGA inhibitors and can increase cellular O-GlcNAcylation level efficiently in cultured cells. Alternatively, RNAi techniques that can down-regulate OGA expression and thus lead to increased cellular O-GlcNAcylation may be used for this purpose in the future.

The moderate change of O-GlcNAcylation with these two compounds ameliorated and aggravated, respectively, ischemia-induced brain damage and motor deficits in mice, suggesting a neuroprotective role of O-GlcNAcylation during cerebral ischemia-reperfusion injury. However, the initial dose of thiamet-G (160 μg/mouse), led to an excessive (>6-fold) elevation of O-GlcNAcylation and more severe brain damage and motor deficits after MCAO in mice. When the thiamet-G dose was lowered to that resulting in a moderate elevation of O-GlcNAcylation, a neuroprotective role against cerebral ischemia insult was observed. The transient, moderate elevation of O-GlcNAcylation during the early phase of cerebral ischemia may thus be neuroprotective, but the excessive elevation of O-GlcNAcylation is detrimental. This double-edged sword phenomenon is actually common in other type of responses to stress and insults, such as neuroinflammation and apoptosis.

To verify the role of O-GlcNAcylation in cerebral ischemia-reperfusion injury, the MCAO-induced brain damage and motor deficits in mice were studied after the neuronal OGT was selectively knocked out. Though these mice did not show a significant reduction of the basal O-GlcNAcylation level in the striatum as compared to the control mice, the ischemia-induced transient elevation of O-GlcNAcylation was markedly prevented and consequently MCAO-induced brain damage and motor deficits were significantly more severe. A very marked increase in the mortality was also observed in the OGT KO mice after MCAO. These results further support the neuroprotective role of O-GlcNAcylation during cerebral ischemic injury.

The dynamic alteration of O-GlcNAcylation during cerebral ischemia-reperfusion injury that we observed in the mouse brain likely occurs in the human brain too. Though it is not possible to determine the time-course changes of O-GlcNAcylation in human ischemic brain tissue, the O-GlcNAcylation level of postmortem human brain tissue sections that contained both ischemic and unaffected tissue was studied from individuals with ischemic stroke. The ischemic tissue was verified by the paler H&E staining and the appearance of macrophages and vacuolization lesion. A marked reduction and even loss of the O-GlcNAc staining was found in the ischemic regions of the human brain tissue sections, which is consistent to the later stages (after six hours post ischemia) of cerebral ischemia in the mouse brain. Indeed, the brain tissue samples were not from the individuals killed by stroke. The small ischemic strokes we studied were extremely unlikely to be fresh strokes that occurred within four hours before the death. Increased O-GlcNAcylation is thus expected in the ischemic human brain tissue if such human tissue samples were available within four hours after stroke.

A dynamic alteration of O-GlcNAcylation (first elevation and then decline) during cerebral ischemia and ischemia-reperfusion injury was demonstrated in mice. O-GlcNAcylation is not merely a response to cerebral ischemia; it serves as a novel regulation of cerebral ischemia-reperfusion injury. The initial and transient elevation of brain O-GlcNAcylation is neuroprotective and helps ameliorate cerebral ischemia-reperfusion injury. These findings suggest the use of O-GlcNAcylation-enhancing compounds as a potential therapeutic strategy for ischemic stroke.

### Example

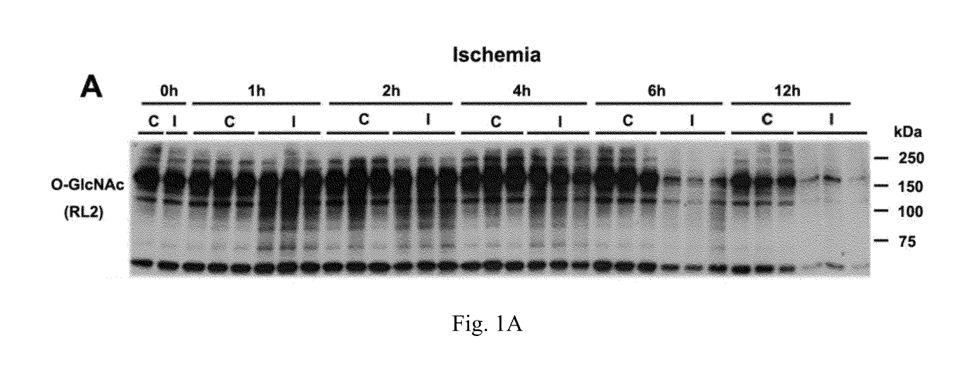

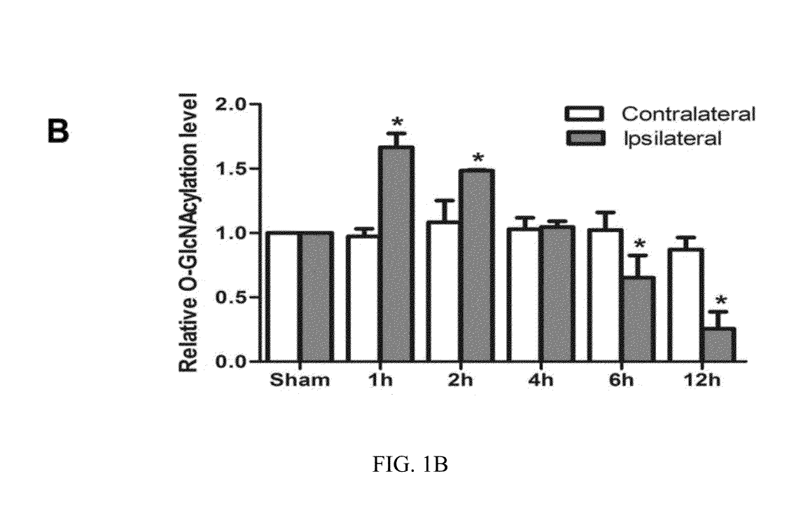

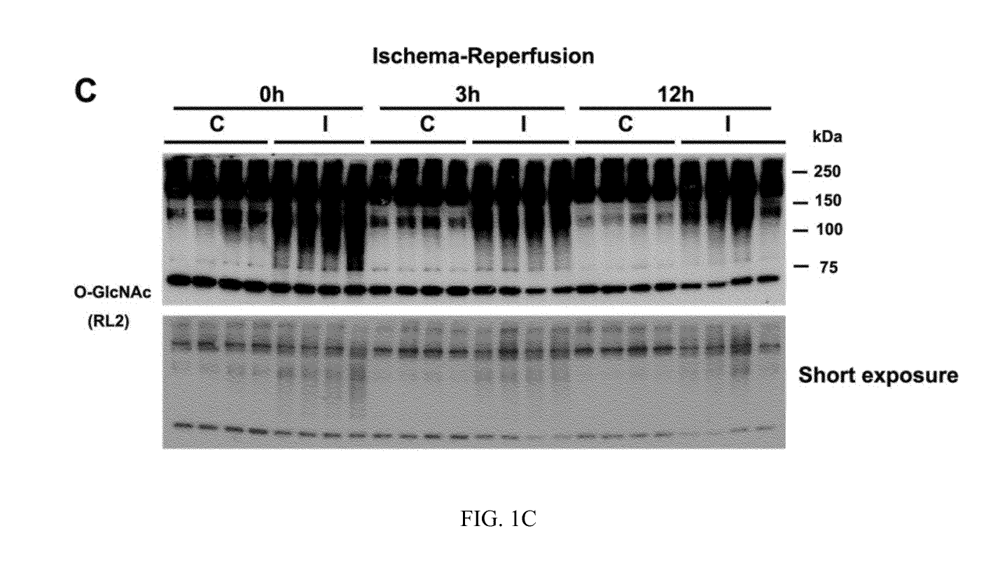

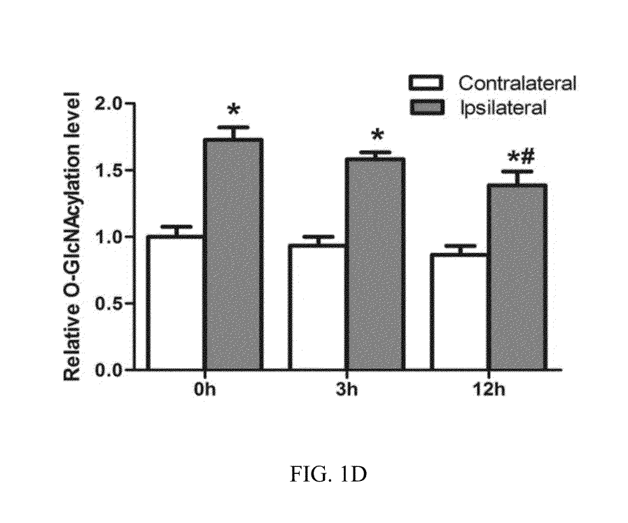

Referring now to the drawings, wherein like reference numerals refer to like parts throughout, there is seen in FIGS. 1-8 the results of an investigation into the use of O-GlcNAcylation treatment for ischemic brain injury. To investigate whether cerebral ischemia causes alterations of brain O-GlcNAcylation, the mouse MCAO model was used and the global protein O-GlcNAcylation level was determined by using Western blots developed with a monoclonal antibody RL2 against O-GlcNAcylated proteins. As seen in FIGS. 1A and 1B, MCAO induced a marked but transient increase in protein O-GlcNAcylation in the ipsilateral cerebral cortex of the mouse brains during 1-2 hrs after occlusion.

After 2 hrs post occlusion, the O-GlcNAcylation level started to reduce to less than 30% of the level of the contralateral side by 12 hrs. There was no significant change in the O-GlcNAcylation level in the contralateral side of the mouse brain except a slight reduction at 12 hrs after MCAO. Similar but more rapid changes of O-GlcNAcylation were seen in the striatum, the core of ischemia induced by MCAO, than the cerebrocortical regions of the ipsilateral brains, whereas the hippocampus showed much smaller changes in O-GlcNAcylation than the cerebral cortex (data not shown). These results indicate a transient elevation followed by a marked reduction in brain O-GlcNAcylation in the ischemic brain tissue. Such a dynamic and regional elevation of protein O-GlcNAcylation was also seen by using double immunofluorescence staining of the brain sections.

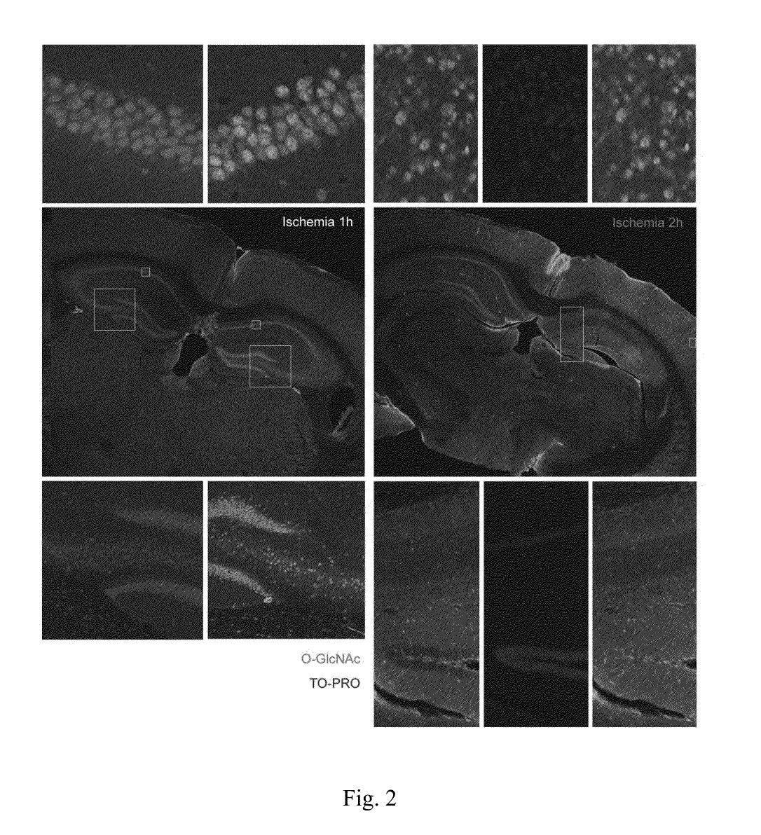

Referring to FIG. 2, a marked increase in neuronal O-GlcNAc staining was seen in the affected brain regions 1-2 hrs post MCAO. The O-GlcNAc staining was most intense in the neuronal nuclei and also in the cytoplasmic compartment of neurons, which is consistent with the nucleocytoplasmic localization of the O-GlcNAcylation modification in the brain.

Reperfusion after cerebral ischemia often induces further tissue damage and is critical to the final outcome of stroke. As a result, protein O-GlcNAcylation was investigated during reperfusion after MCAO for two hrs. The elevated brain O-GlcNAcylation lasted for a much longer time during reperfusion as compared with permanent cerebral ischemia. Although the O-GlcNAcylation level slightly decreased during the first 12 hrs of reperfusion, it was still much higher in the ipsilateral side than the contralateral side up to 12 hrs after reperfusion, as seen in FIGS. 1C and 1D. Therefore, while brain O-GlcNAcylation is increased only transiently for 1-2 hrs after ischemia, this increase lasts many hours after reperfusion.

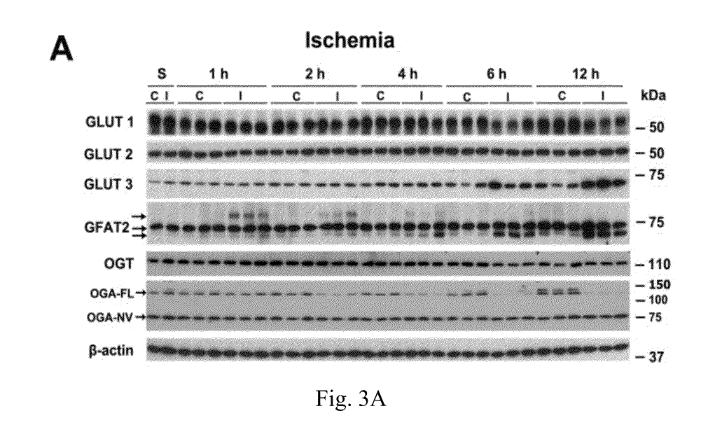

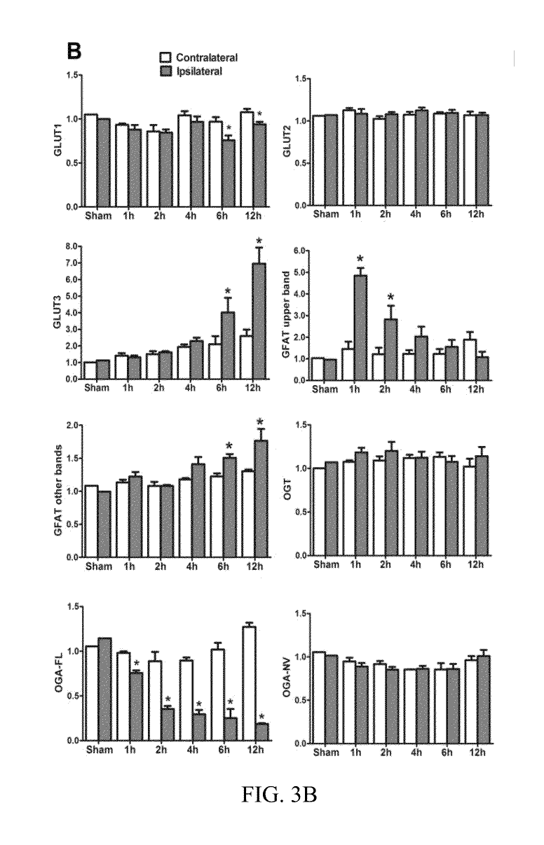

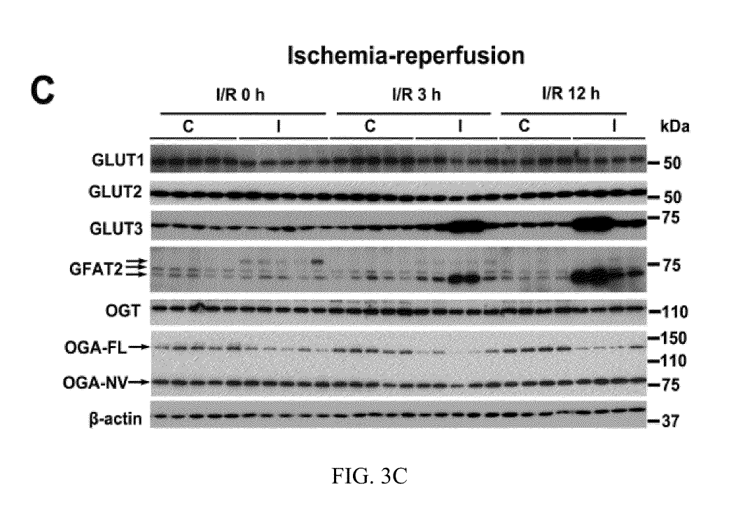

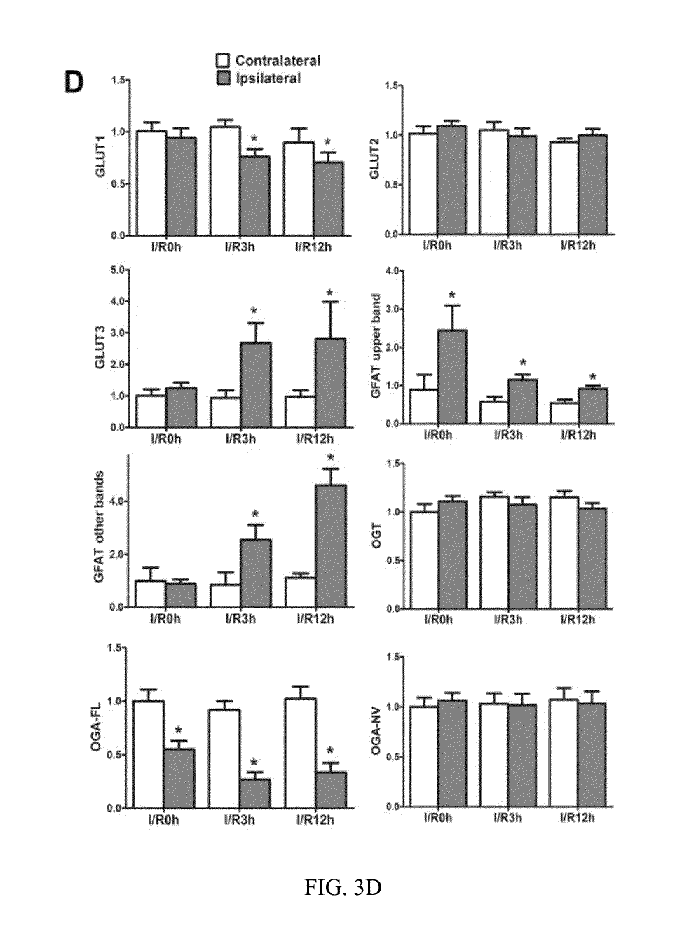

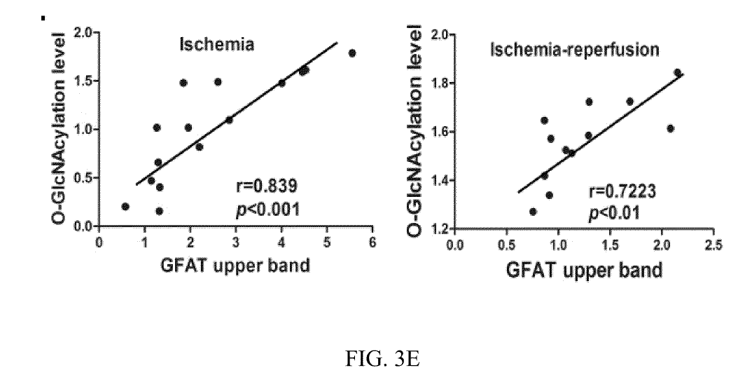

To learn the possible molecular mechanism involved in the dynamic alterations of brain protein O-GlcNAcylation during cerebral ischemia, major brain glucose transporters (GLUTs) were investigated, which determine intracellular glucose level and thus affect O-GlcNAcylation because UDP-GlcNAc (the GlcNAc donor of protein O-GlcNAcylation) is synthesized from intracellular glucose, as well as several enzymes that control or regulate O-GlcNAcylation. Among the major brain GLUTs, a slight decrease in the level of GLUT1 was found, the major GLUT located at the endothelial cells of the blood-brain barrier, after ischemia for 6-12 hrs and after 2 hrs of ischemia followed by reperfusion for 3 hrs or longer, as seen in FIGS. 3A through 3D. The level of GLUT2, a bidirectional GLUT located on the glial cell membrane in the brain, was not changed after cerebral ischemia or ischemia-reperfusion for up to 12 hrs. The level of GLUT3, the major neuronal GLUT responsible for transporting glucose from the extracellular space into the neuron, was found to be increased markedly at the later hours after ischemia or ischemia-reperfusion injury. These results suggest that the transient elevation and the following decline of brain protein O-GlcNAcylation do not result from corresponding glucose influx because the major brain GLUTs did not alter in the same time-dependent manner.

The rate-limiting enzyme of the HBP in the brain is glutamine/fructose-6-phosphate amidotransferase 2 (GFAT2), which controls the HBP flux and determines the production of UDP-GlcNAc. GFAT2 normally displays as a 72-kDa protein band in SDS-PAGE as seen in FIGS. 3A and 3C (middle arrow of the GFAT2 blots). The level of this GFAT2 band was increased 6 hrs or longer after ischemia and 3-12 hrs after reperfusion following ischemia, as seen in FIGS. 3A through 3D. However, a higher apparent molecular weight band (˜77 kDa) of GFAT2 was seen in the ipsilateral side of the MCAO mouse brains at 1 and 2 hrs after ischemia as seen in FIGS. 3A and 3B, which coincided exactly with the dynamic elevation of brain protein O-GlcNAcylation, as seen in FIG. 1. Because an up-shift of apparent molecular weight in SDS-PAGE is a common phenomenon of protein phosphorylation, this GFAT2 upper band is likely the phosphorylated GFAT2 that has been reported to have a higher enzymatic activity than the non-phosphorylated counterpart.

To verify this possibility, the brain homogenates were incubated with alkaline phosphatase in vitro before SDS-PAGE and it was found that the level of the upper GFAT2 band was markedly reduced after the treatment with alkaline phosphatase, supporting that the upper band is the phosphorylated GFAT2.

To verify if the appearance/elevation of the upper GFAT2 band is involved in the increase in O-GlcNAcylation, a correlation analysis was performed between the level of the GFAT2 upper band and the O-GlcNAcylation level. These two were strongly correlated, as seen in FIG. 3E, suggesting that the elevated GFAT2 phosphorylation/activation attributes to the dynamic elevation of O-GlcNAcylation in the mouse brain after ischemia.

Because OGT (that catalyzes O-GlcNAcylation reaction) and OGA (that removes O-GlcNAc from proteins) are the enzymes that control protein O-GlcNAcylation level directly, their levels were determined in the ischemic brain tissue. The OGT level was unchanged in the mouse brains during ischemia and ischemia-reperfusion injury, as seen in FIGS. 3A through 3D. OGA has two splicing variants—the cytosolic full-length (OGA-FL, 120-130 kDa) and the nuclear variant (OGA-NV, 75 kDa). The cytoplasmic OGA-FL level was decreased dramatically in a time-dependent manner under the conditions of both ischemia and ischemia-reperfusion injuries, but the nuclear OGA-NV remained unchanged, as seen in FIGS. 3A through 3D. These results exclude the critical role of OGT and OGA in the transient elevation of O-GlcNAcylation during early stage of ischemia and suggest that OGA may be degraded in the cytosolic compartment, but not in the nucleus, during ischemia and ischemia-reperfusion injuries.

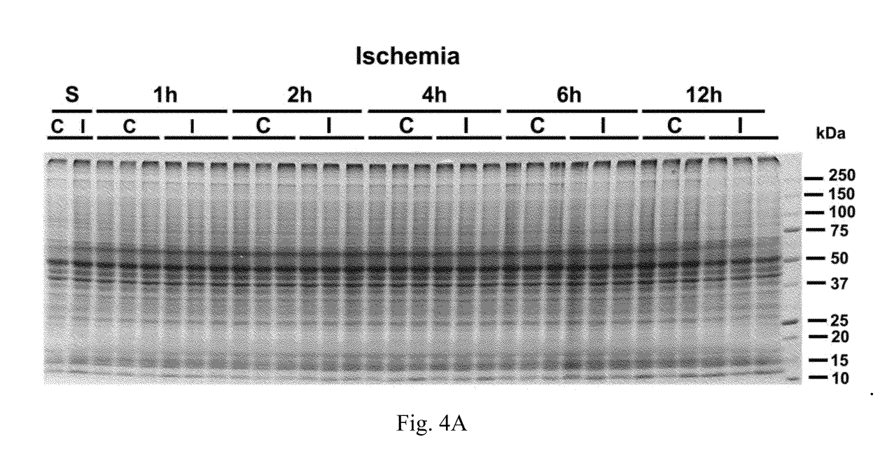

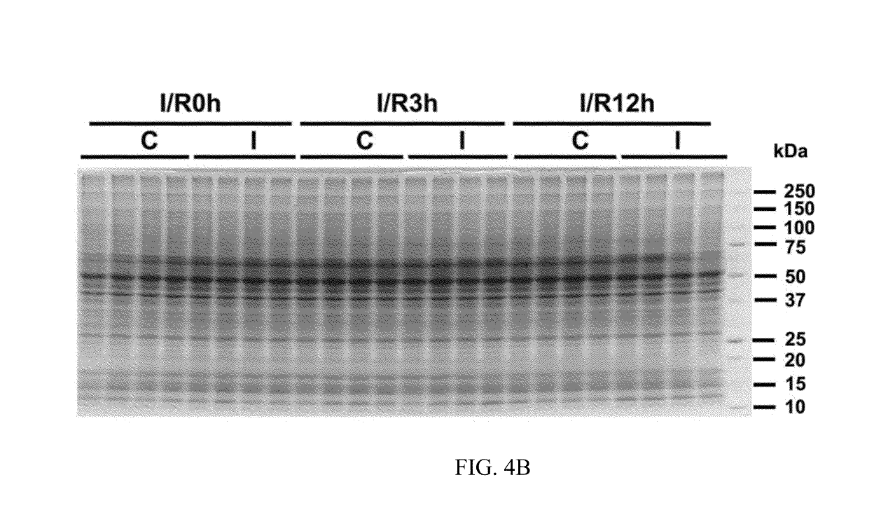

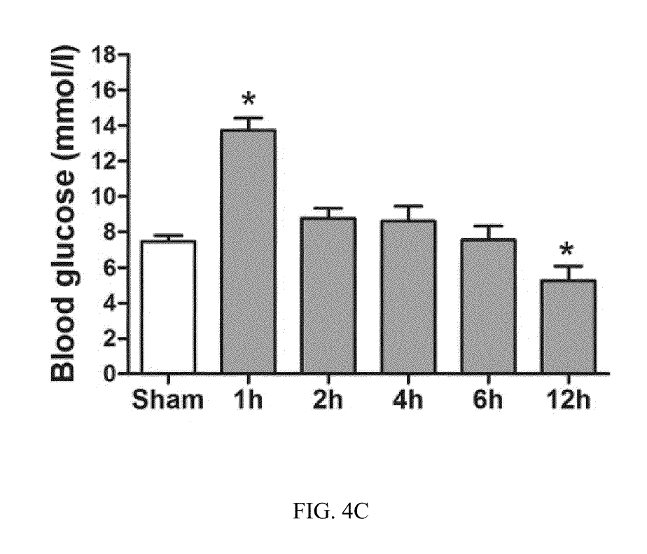

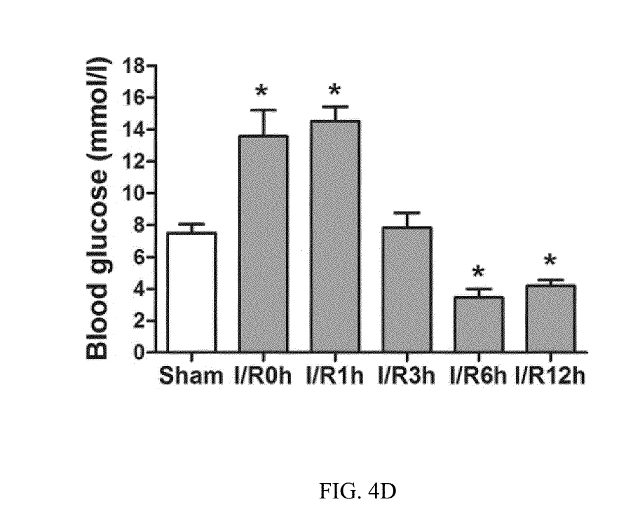

To investigate whether the marked, time-dependent reduction of OGA-FL is a result of marked global cerebral protein degradation in the cytoplasmic compartment, the gels were stained with Coomassie brilliant blue and found marked global protein degradation only 12 hrs after ischemia or reperfusion, as seen in FIGS. 4A and 4B. These results suggest that OGA-FL is selectively degraded in the brain during ischemia-reperfusion injury.

The blood glucose level of mice was also determined during ischemia-reperfusion injury and observed that it markedly increased one hour after ischemia, as seen in FIG. 4C, and reperfusion following 2-hr-ischemia, as seen in FIG. 4D. This elevation of blood glucose is consistent with the body's stress response to surgery and might also contribute to the elevated brain O-GlcNAcylation at this time period. The blood glucose level was decreased 6-12 hrs after the surgery, as seen in FIGS. 4C and 4D, probably due to the reduced food intake after surgery.

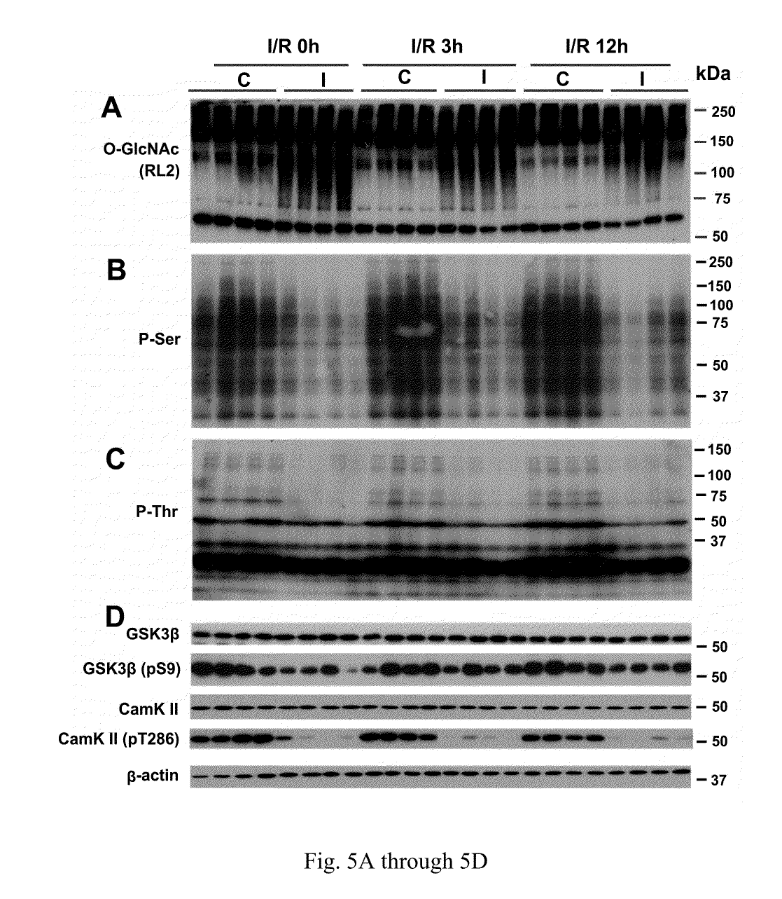

O-GlcNAcylation often regulates phosphorylation of proteins and, in some proteins, these two posttranslational modifications regulate each other in a reciprocal manner. To investigate the correlation of global O-GlcNAcylation with global protein phosphorylation during brain ischemia-reperfusion injury, global protein phosphorylation was analyzed by Western blots developed with anti-phosphoserine, as seen in FIG. 5B, and anti-phosphothroenine, as seen in FIG. 5C. While O-GlcNAcylation was marked increased, protein phosphorylation, especially as determined by anti-phosphoserine, was markedly decreased in the ipsilateral side of the ischemia-reperfusion brains, as seen in FIGS. 5A through 5C. Marked decrease in phosphorylation was also seen at serine 9 of glycogen synthase kinase 3β (GSK-3β) and threonine 286 of calcium/calmodulin-depenedent protein kinase II (CaMK-II), as seen in FIG. 5D. These results suggest a reciprocal relationship between global O-GlcNAcylation and phosphorylation in the brain during ischemia-reperfusion injury.

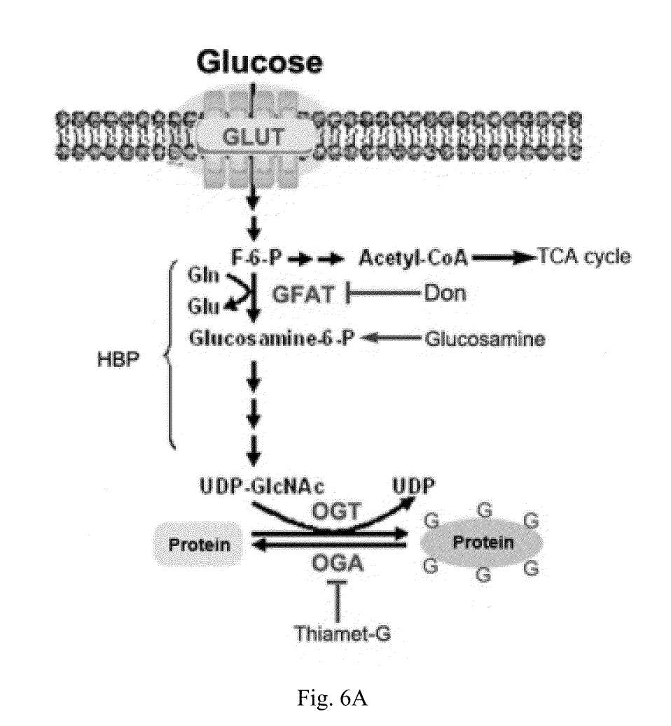

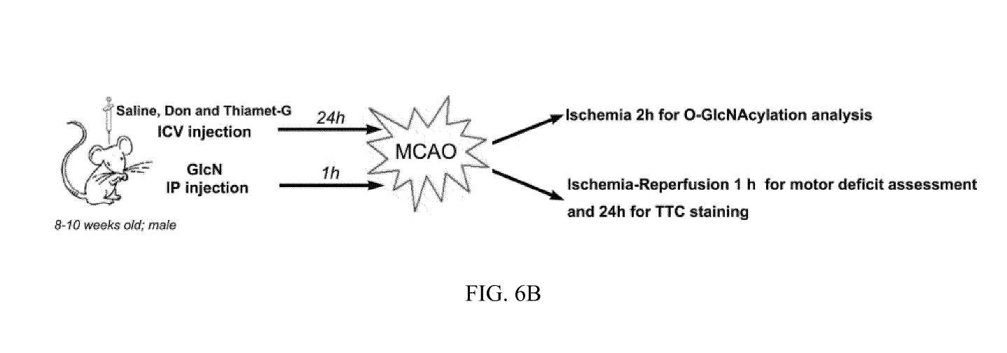

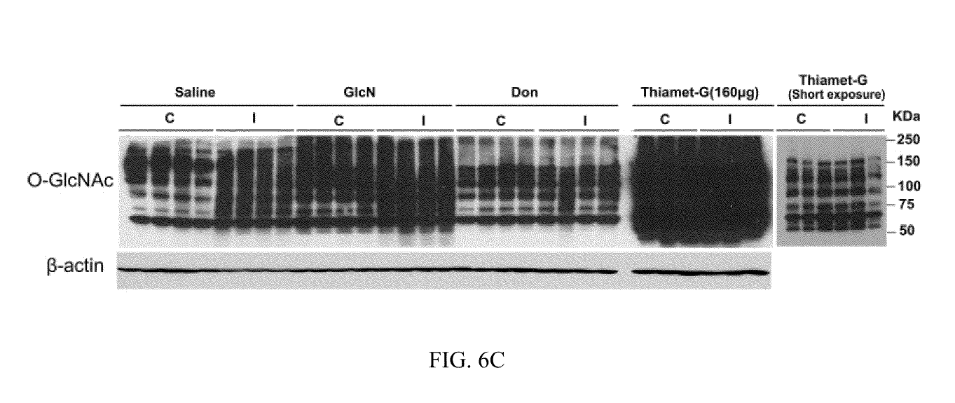

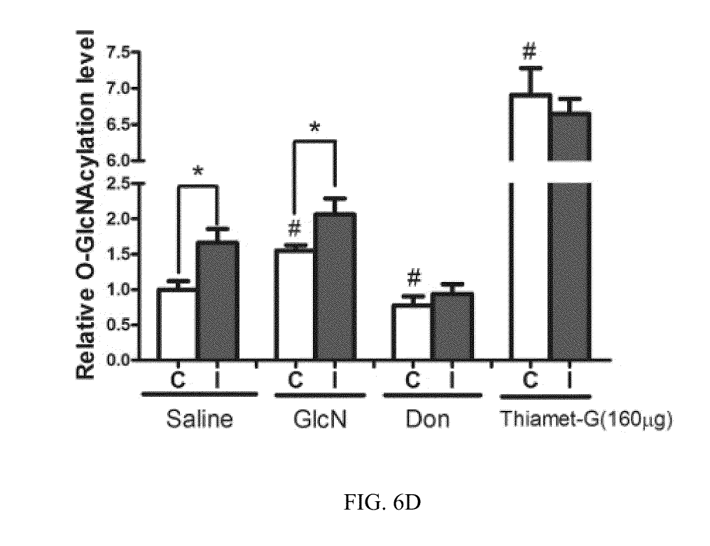

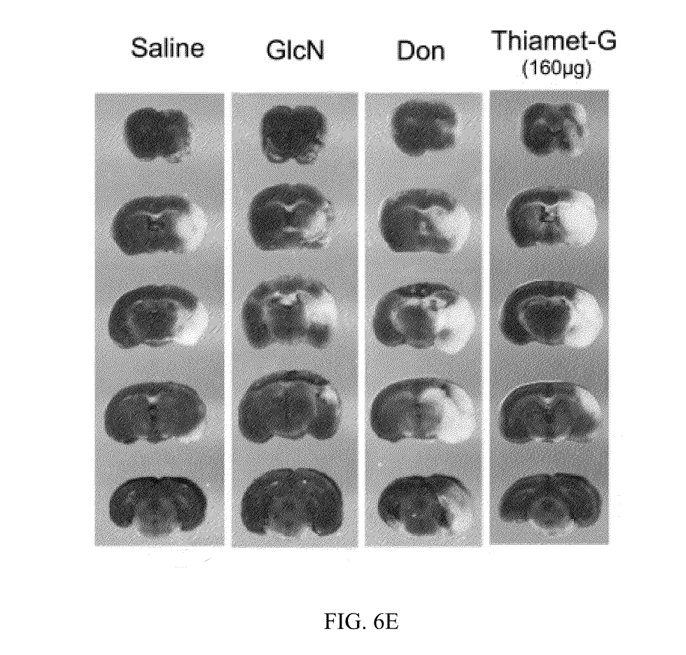

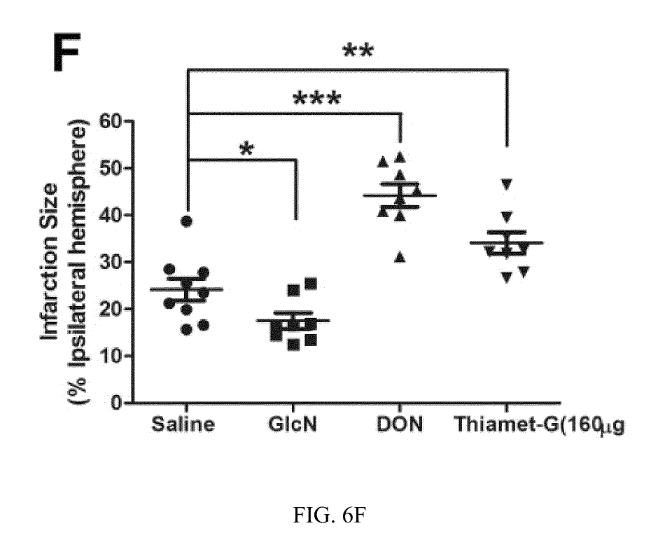

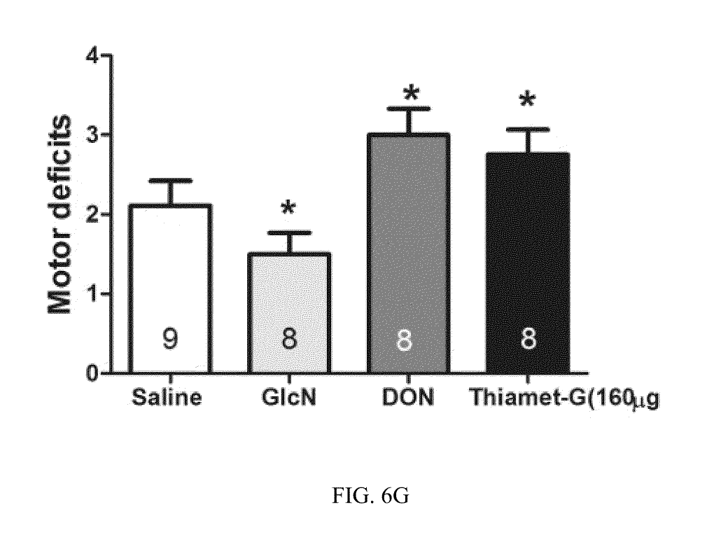

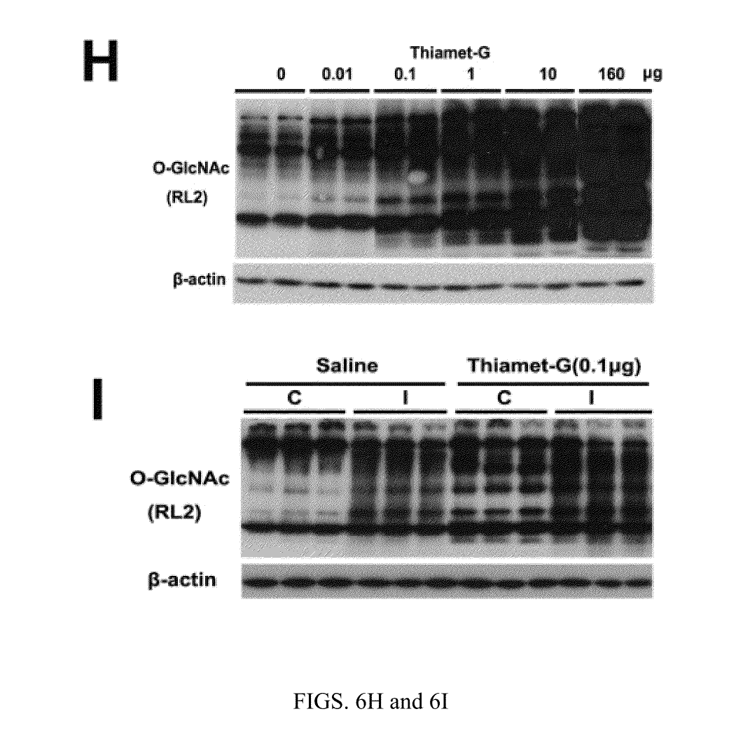

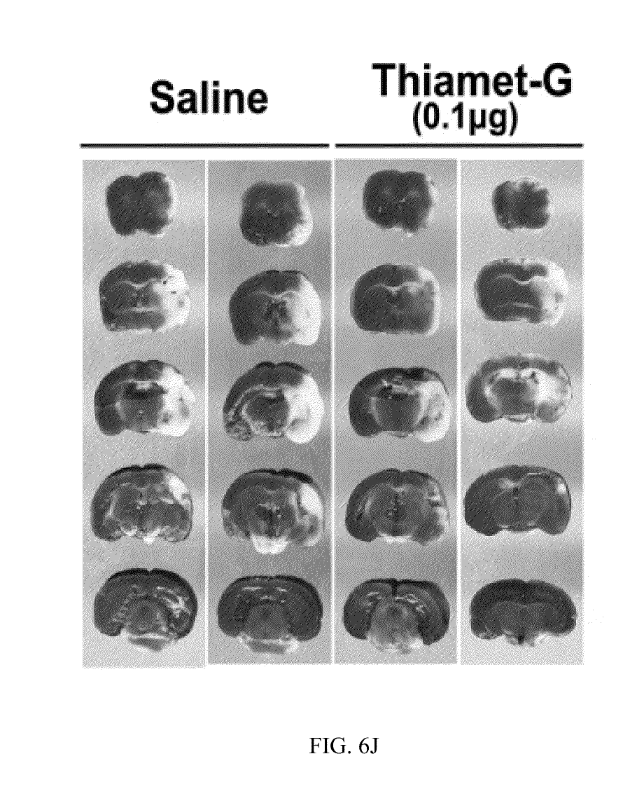

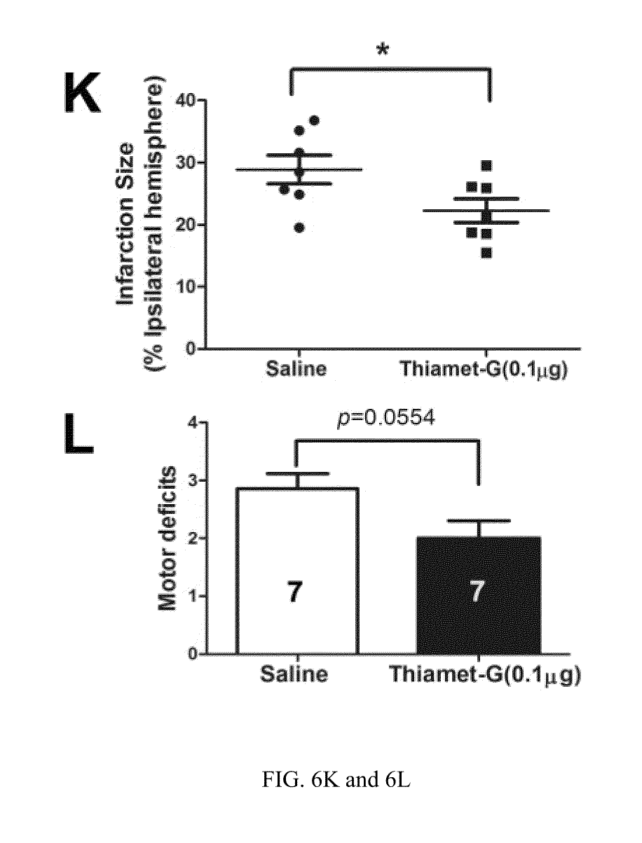

To investigate whether the alteration of O-GlcNAcylation affects cerebral ischemia-reperfusion injury and recovery, the brain O-GlcNAcylation level was manipulated pharmacologically, as seen in FIG. 6A, and then studied for the impact on motor function of the mice and the infarct size, as seen in FIG. 6B. Brain O-GlcNAcylation was reduced by treating mice with 2.6 μg/mouse 6-diazo-5-oxonorleucine (DON) (GFAT inhibitor) and elevation of brain O-GlcNAcylation with 10 mg/mouse glucosamine (GlcN, which increase the HBP flux bypassing GFAT) or 160 μg/mouse thiamet-G (OGA inhibitor). Western blots of brain samples collected 2 hrs after ischemic insult showed the expected changes in O-GlcNAcylation of brain proteins, as seen in FIGS. 6C and 6D.

The elevation of O-GlcNAcylation prior to ischemia with glucosamine reduced both the motor deficits, as determined one hour after ischemia-reperfusion, and the infarct size, as determined 24 hrs after ischemia-reperfusion, as seen in FIGS. 6E through 6G. In contrast, reduction of O-GlcNAcylation prior to ischemia with DON increased both the motor deficits and the infarct size. However, a very dramatic increase in O-GlcNAcylation (>6-fold) with thiamet-G did not reduce ischemia-induced motor deficits or infarct size, as seen in FIGS. 6E through 6G). Instead, it worsened the motor deficits and infarct size.

To elucidate whether the different and opposite effects between glucosamine and thiamet-G, both of which led to elevated O-GlcNAcylation, on ischemia-induced brain injury are drug-specific phenomenon or due to the huge difference in the O-GlcNAcylation levels produced, thiamet-G concentrations were titrated in order to find a concentration that produced a similar level of O-GlcNAcylation as with the glucosamine treatment. 0.1 μg/mouse thiamet-G (icy injection) produced a comparable elevation of O-GlcNAcylation as did with 10 mg glucosamine/mouse (i.p. injection), as seen in FIGS. 6H and 6I. This dose of thiamet-G reduced ischemia-induced motor deficits and infarct size, as seen in FIGS. 6J through 6L. These results suggest that a moderate elevation of O-GlcNAcylation (<3-fold increase) is neuroprotective, whereas excessive elevation of O-GlcNAcylation (>6-fold) may further damage ischemia-reperfusion-induced brain injury.

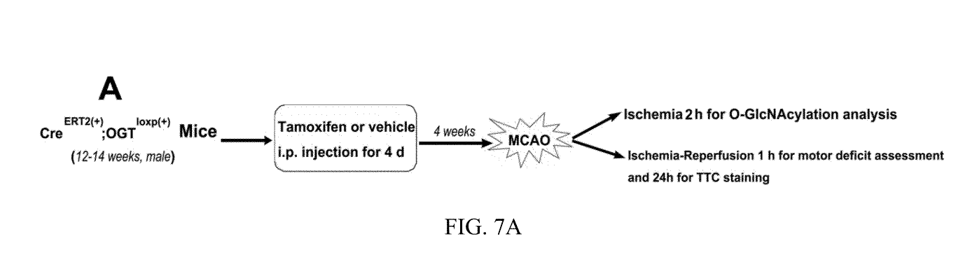

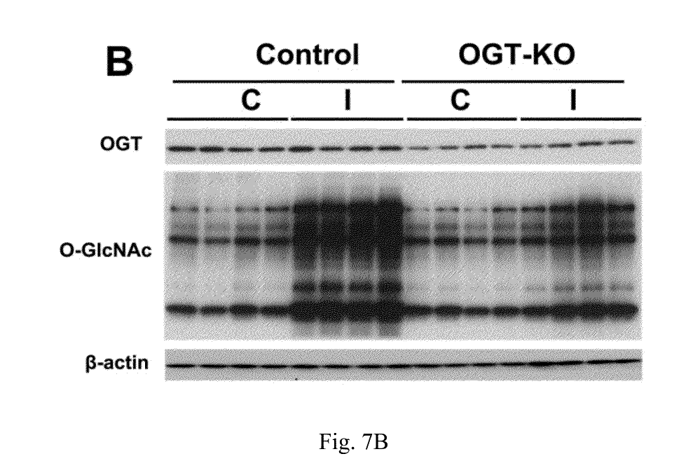

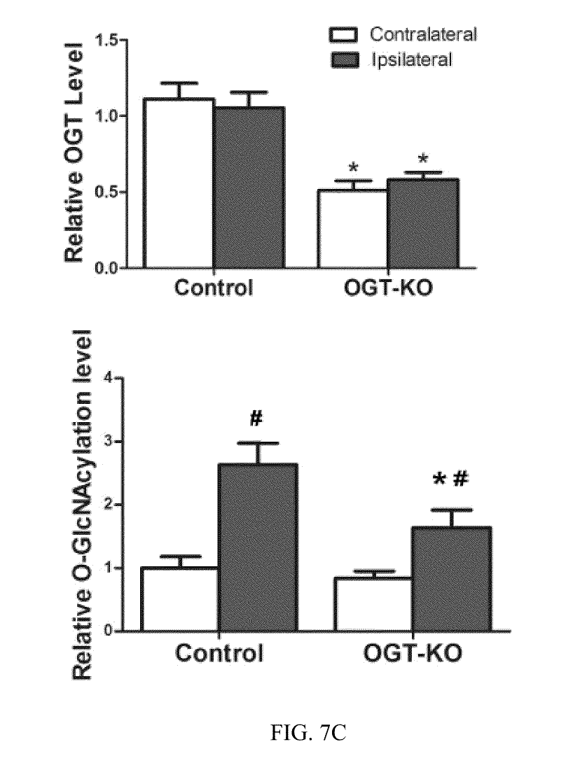

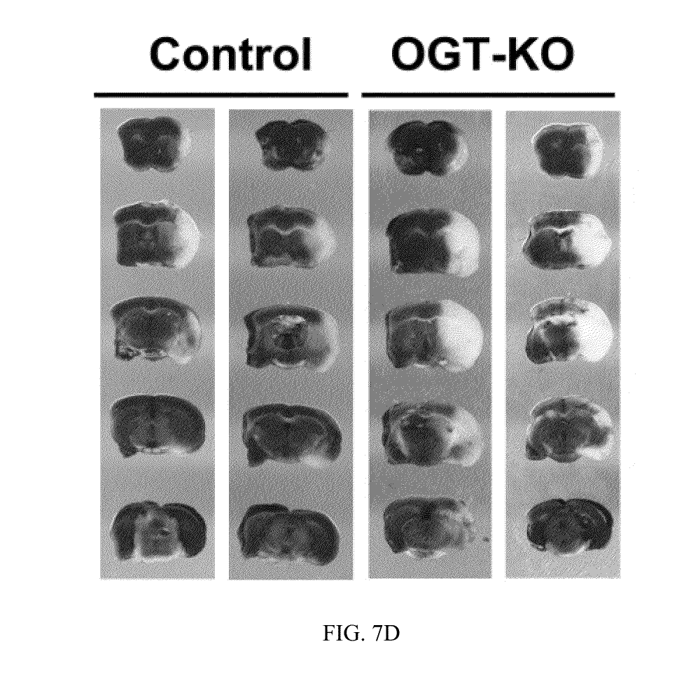

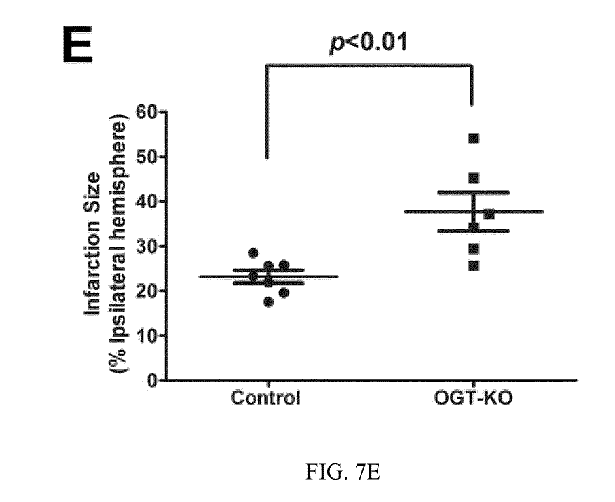

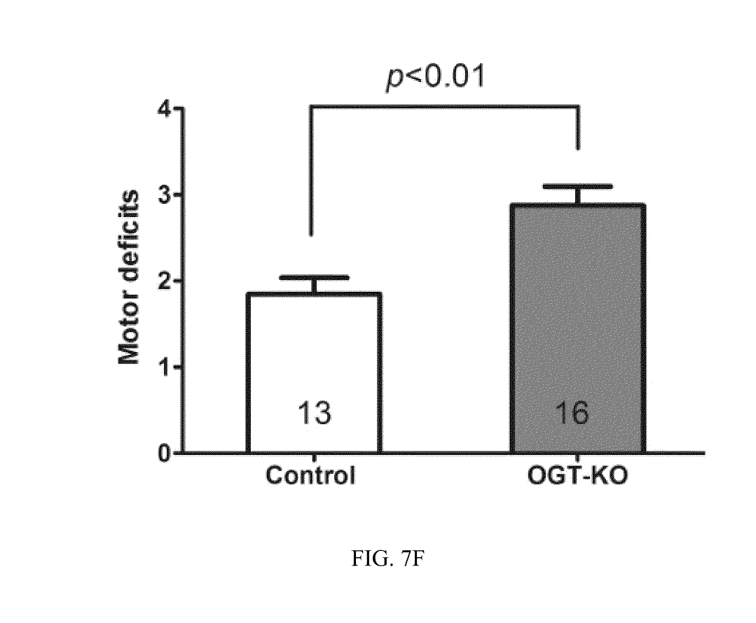

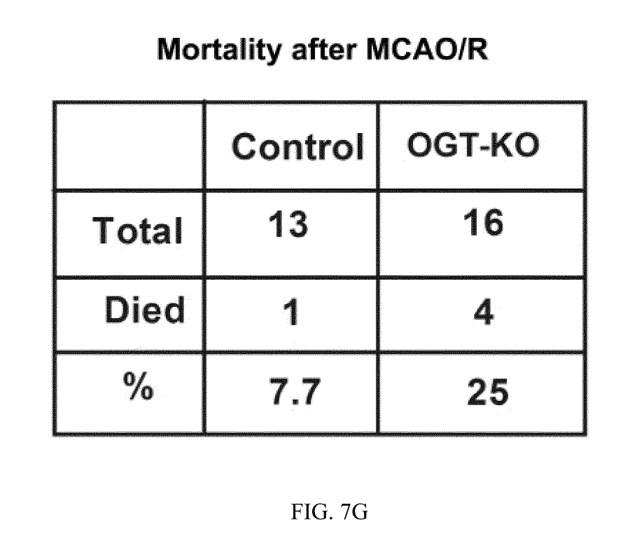

To confirm the role of O-GlcNAcylation on cerebral ischemia-reperfusion injury, a neuron-specific brain OGT knockout (KO) mouse model was developed in which the OGT gene was floxed by two loxP sites and the CRE expression was induced by tamoxifen administration and controlled by the CaMK-IIα promoter. The mice were subjected to MCAO four weeks after induction of neuronal OGT KO with tamoxifen injection for consecutive four days. The OGT and O-GlcNAcylation levels were determined in the striatum (the major ischemia site after MCAO) by Western blots two hours after ischemia, motor deficits were assessed after two-hour ischemia followed by one-hour reperfusion, and brain tissue damage was detected 24 hrs after reperfusion in mice, as seen in FIG. 7A. As expected, we observed approximately 50% reduction in the level of OGT in the striatum of the OGT KO mice as compared to the mice without KO induction, as seen in FIGS. 7B and 7C, confirming the success of inducing OGT KO four weeks after tamoxifen injection. The remaining OGT was likely from non-neuronal cells of the brain tissue. In the ischemic brain tissue, the protein O-GlcNAcylation was markedly increased two hours after MCAO in the control mice, but this increase was largely prevented in the neuronal OGT KO mice, as seen in FIGS. 7B and 7C. When the elevation of O-GlcNAcylation was partially diminished, the MCAO-induced brain infarct size was larger, as seen in FIGS. 7D and 7E, and the motor deficits were more severe, as seen in FIG. 7F, in the neuronal OGT KO mice than in control mice. MCAO also produced more mortality in the neuronal OGT KO mice (25%) than in the control mice (7.7%), as seen in FIG. 7G. These results suggest that prevention of the dynamic elevation of O-GlcNAcylation aggravates cerebral ischemia-reperfusion injury and thus further support the protective role of O-GlcNAcylation during ischemia-reperfusion injury.

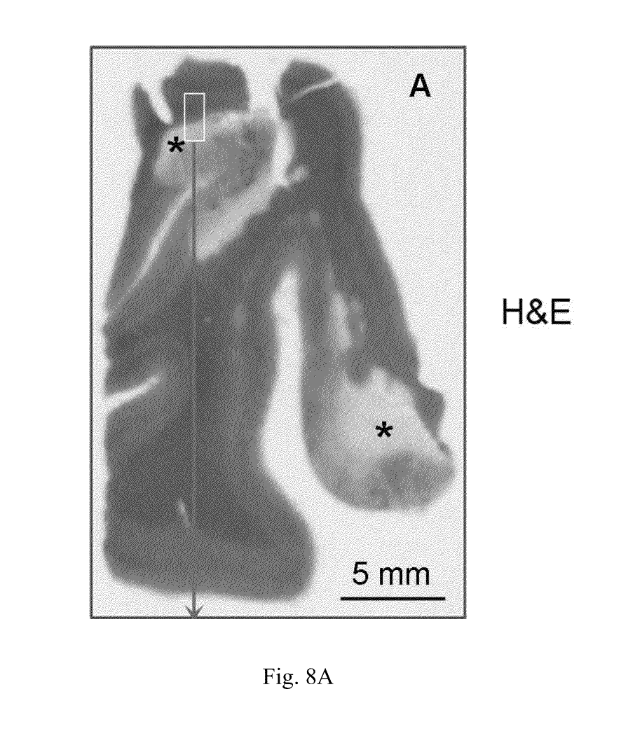

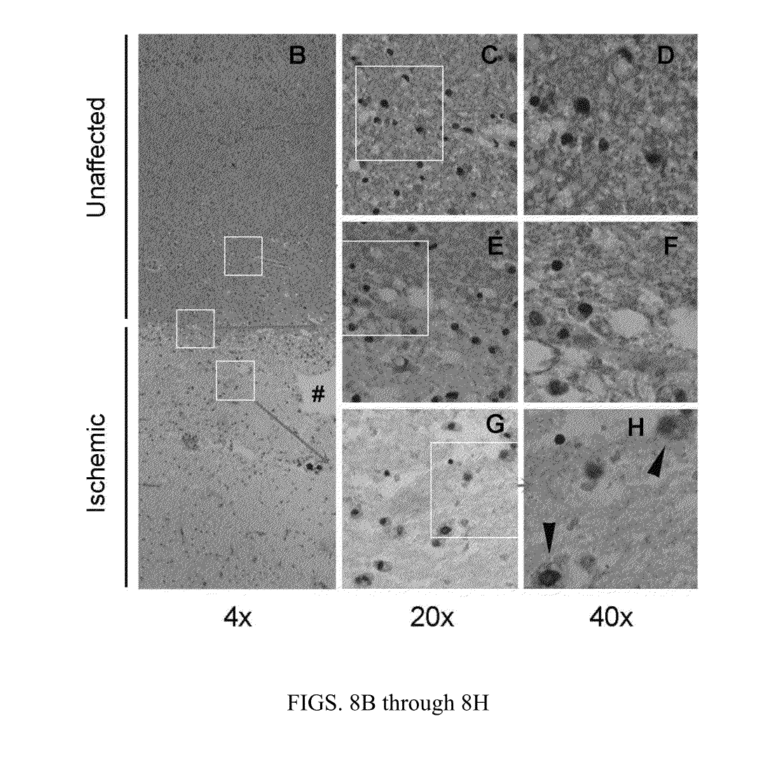

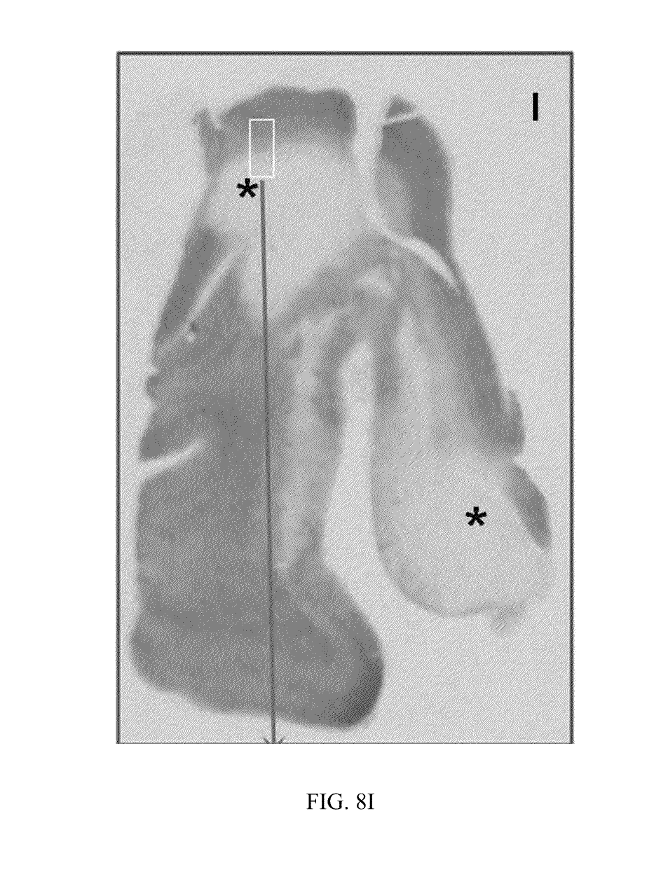

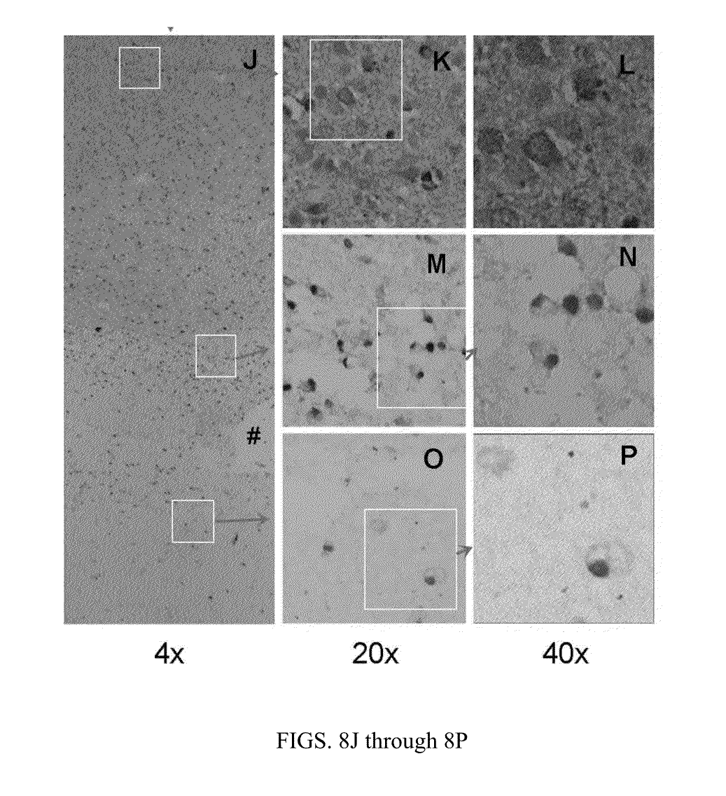

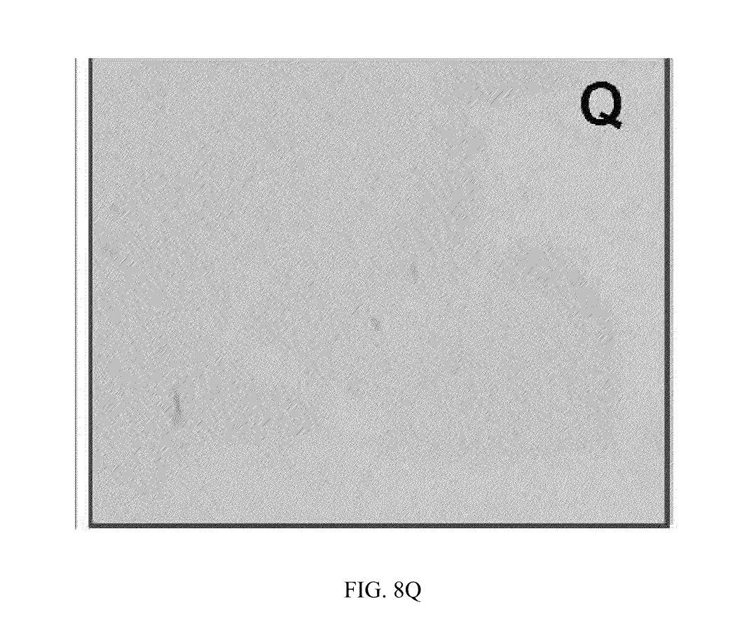

To learn whether the alteration of brain protein O-GlcNAcylation observed in MCAO mice is consistent with human cases with ischemic stroke, the adjacent paraffin sections of four postmortem human brains were stained, which all contained both the ischemic and unaffected areas of brain tissue. The ischemic area of brain tissue was confirmed by histological examination of the tissue sections stained with hematoxylin and eosin (H&E). It is well established that the ischemic brain tissue has much paler H&E staining than the normal healthy brain tissue. This approach was used to localize the ischemic brain area, as seen in FIGS. 8A and 8B, which was confirmed by the loss of neuronal cells as well as appearance of macrophages and vacuolization, as seen in FIGS. 8E through 8H. Comparison between the H&E staining, as seen in FIG. 8A, and O-GlcNAc staining, as seen in FIG. 8I, of the adjacent section indicated marked weaker immunostaining of O-GlcNAc in the ischemic areas as marked by asterisks. The RL2 staining was specific because its omission from the primary antibody solution, as a control, eliminated the DAB staining, as seen in FIG. 8Q. Under higher magnifications, O-GlcNAc was seen both in the nuclei and cell bodies and neurites, as seen in FIGS. 8K and 8L, in the normal cerebral cortex, which is consistent with the previous observations. However, at the border region between the normal and the ischemic core cortex (penumbra), the O-GlcNAc staining was found to be condensed in the nuclei with marked decrease or loss of the O-GlcNAc staining in the cytoplasmic compartment, as seen in FIGS. 8M and 8N. Vacuolization lesion was also obvious with O-GlcNAc staining at the border region. The O-GlcNAc staining was almost lost in the ischemic regions, leaving positive staining in the nuclei of some glial cells and macrophages, as seen in FIGS. 8O and 8P. These results indicate a marked decrease of O-GlcNAcylation after ischemia in the human brain.

The appropriate dosages for human patients are likely as follows: glucosamine, 1-10 mg/day via intravenous injection; thaimet-G, 0.1-1 mg/day via intravenous injection or 1-10 mg/day via oral administration; and GlcNAcstatin G, 0.02-0.2 mcg/day via intravenous injection or 0.2-2 mcg/day via oral administration. It should be recognized by those of skill in the art that dosages may be determined according to known procedures after sufficient testing in compliance governmental regulations, such as those promulgated by the U.S. Food and Drug Administration. The appropriate dosage should produce a moderate elevation of O-GlcNAcylation (<3-fold increase) as this change is neuroprotective. Thus, the target level of brain O-GlcNAcylation could be 1-3-fold higher than the endogenous level for the treatment of brain ischemic injury. For best results, treatment should be administered within 1-3 hours after brain ischemic injury, and treatment should continue for 2-3 days.

### Materials and Methods

### Reagents and Antibodies

Primary antibodies used in this Example are listed in Table 1. Peroxidase-conjugated anti-mouse and anti-rabbit IgG were obtained from Jackson ImmunoResearch Laboratories (West Grove, Pa.). Alexa 488-conjugated goat anti-mouse IgG and TO-PRO-3 iodide (642/661) were from Life Technologies (Grand Island, N.Y.). The enhanced chemiluminescence (ECL) kit was from Thermo Scientific (Rockford, Ill.). Other chemicals were from Sigma (St. Louis, Mo.).

TABLE 1
primary antibodies used in this study

| Antibody | Type | Specificity | Source/reference |
|---|---|---|---|
| RL2 | Mono- | 0-GlcNAc | Thermo Scientific |
| CTD110.6 | Mono- | O-GlcNAc | Covance, Emeryville, CA |
| GLUT1 | Poly- | GLUT1 | Millipore, Temecula, CA |
| GLUT2 | Poly- | GLUT2 | Milipore, Billerica, MA |
| GLUT3 | Poly- | GLUT3 | Santa Cruz Biotechnology, CA |
| GFAT2 | Poly- | GFAT2 | Santa Cruz Biotechnology |
| (H-300) | | | |
| OGA | Poly- | OGA | |
| OGT | Poly- | OGT | Sigma, St. Lious, MO |
| pSer | Poly- | Phosphoserine | Invitrogen, Carlsbad, CA |
| pThr | Poly- | Phosphothreonine | Invitrogen |
| GSK-3β | Poly- | GSK-3β | Cell Signaling Technology |
| GSK-3β | Poly- | P-GSK-3β (Ser9) | Cell Signaling Technology |
| (pS9) | | | |
| CaMK-II | Poly- | CaMK-II | EMD Millipore, MA |
| CaMK-II | Poly- | P-CaMK-II | Promega, WI |
| (pT286) | | (Thr286) | |
| β-actin | Mono- | β-actin | Sigma |

### Human Brain Tissue

Paraffin-embedded human brain tissue sections were obtained from the Banner Sun Health Research Institute Brain and Body Donation Program (Sun City, Ariz.). Cerebral ischemia in these sections was confirmed histologically. The use of the tissue was in accordance with the National Institutes of Health guidelines and was approved by our institutional review board.

### Animals

All strains of mice were initially purchased from the Jackson Laboratory (New Harbor, Me.) and bred in our animal colony. Mice were housed (4-5 animals per cage) with a 12/12-hr light/dark cycle and with ad libitum access to food and water. The housing, breeding, and animal experiments were in accordance with the approved protocol from the Institutional Animal Care and Use Committee and with the PHS Policy on Human Care and Use of Laboratory animals (revised Mar. 15, 2010). Unless specified (see below), male C57BL/6J mice weighing 25-30 g were used for this study.

The Camk2aCre(+)-Ogtloxp(+)/loxp(+) mice were generated by crossing the hemizygous B6.129S6-Tg (Camk2a-cre/ERT2)1Aibs/J Cre-expressing mice with the B6.129-OgttmGwh/J OGT-floxed mice, both of which were purchased from the Jackson Laboratory and back crossed for at least 10 generations in an animal colony. The male neuronal OGT KO in the Camk2aCre(+)-Ogtloxp(+)/loxp(+) mice was induced by intraperitoneal injection (i.p.) of tamoxifen (75 mg/kg/day for 4 consecutive days) or, as a control, vehicle (corn oil containing 10% ethanol). The mice were subjected to MCAO or sham surgery 4 weeks after tamoxifen injection.

Intracerebroventricular (icv) Injection

Mice were first anesthetized using 2.5% avertin (2,2,2-tribromoethanol, Sigma-Aldrich), a commonly used anesthetic for mice which produces a wide anesthetic window, and then restrained onto a stereotaxic apparatus. The bregma coordinates used for injection were: −1.0 mm lateral, −0.3 mm posterior, and −2.5 mm below. Each mouse received a single icy injection of the indicated drug in 3.0 μl 0.9% saline or, as a control, 3.0 μl 0.9% saline alone into the right ventricle of the brain. The injection was administered slowly over a period of 5 min, and the needle was retained for another 10 min before removal.

### Middle Cerebral Artery Occlusion (MCAO) and Reperfusion

The MCAO surgery was performed as described previously. Briefly, mice were anesthetized with intraperitoneal injection of 2.5% Avertin. Through a ventral midline incision, the right common carotid artery, internal carotid artery and external carotid artery were surgically exposed. A 6-0 nylon suture with silicon coating (Doccol Corporation, Redlands, Calif.) was inserted into the internal carotid artery through the external carotid artery stump and was gently advanced to occlude the middle cerebral artery. To obtain blood reperfusion, the occluding filament was withdrawn after occlusion for 2 hrs. Cerebral blood flow was monitored by Laser-Doppler flowmetry and only those mice with 90% of blood flow blockade during MCAO and 85-95% recovery of blood flow during reperfusion were used for further experiments. The Sham-operated mice underwent identical surgery but the suture was not inserted. At indicated time points post-ischemia-reperfusion, mice were sacrificed.

### Evaluation of Neurological Deficits and Brain Infarct Size

For determining neurological deficits, neurological assessment was performed on mice post-ischemia-reperfusion surgery using a 5-point scoring system, as follows: 0, normal motor function; 1, flexion of torso and contralateral forelimb when mouse was lifted by the tail; 2, circling to the contralateral side when mouse held by the tail on a flat surface but normal posture at rest; 3, leaning to the contralateral side at rest; 4, no spontaneous motor activity. Neurological assessment was performed by an investigator blinded to the experimental treatments.

Infarct sizes in the mouse brains were determined after staining with 1% 2,3,5-triphenyltetrazolium chloride (TTC) by using the Image Pro Plus software (Media Cybernetics, Silver Spring, Md.). Briefly, 24 hrs after reperfusion, the mice were killed and the brains were cut into 2-mm-thick coronal slices with a brain-cutting matrix (ASI Instruments, Warren, Mich.). After incubation in the TTC solution at 37° C. for 30 min, the TTC-stained brain slices were mounted on dry paper and photographed with a digital camera. The presence of infarction was determined by the areas that lack TTC staining. The infarct volume was expressed as a percentage area of the ipsilateral hemisphere.

### Western Blots

Mouse brain tissue was homogenized in pre-chilled buffer containing 50 mM Tris-HCl (pH 7.4), 50 mM GlcNAc (inhibitor of OGA that catalyzes the removal of O-GlcNAc from a protein), 20 μM UDP (inhibitor of OGT), 2.0 mM EGTA, 2 mM Na₃VO₄, 50 mM NaF, 0.5 mM AEBSF, 10 μg/ml aprotinin, 10 μg/ml leupeptin and 4 μg/ml pepstatin A. Protein concentrations of the homogenates were determined by using Pierce 660 nm Protein Assay kit (Thermo Fisher Scientific Inc.). The samples were resolved in 10% sodium dodecyl sulfate polyacrylamide gel electrophoresis (SDS-PAGE) and electro-transferred onto Immobilon-P membrane (Millipore, Bedford, Mass.). The blots were then probed with a primary antibody (see Table 1), washed and then incubated with a corresponding HRP-conjugated secondary antibody. The protein-antibody complexes were visualized by using the Pierce ECL Western Blotting Substrate (Thermo scientific) and exposed to a HyBlot CL autoradiography film (Denville Scientific, Inc., Metuchen, N.J.). Specific immunostaining was quantified by using the Multi Gauge software V3.0 (Fuji Photo Film Co., Ltd).

### Immunohistochemistry

Frozen mouse brain sections (40-μm thick) were first blocked with 5% normal goat serum in TBS for 30 min, followed by incubation with a primary antibody solution in TBS containing 5% goat serum and 0.1% Triton X-100 at 4° C. overnight. After washing with TBS, the sections were incubated with Alexa 488-conjugated goat anti-mouse IgG (1:1000) plus TO-PRO in TBS containing 5% goat serum and 0.1% Triton X-100 at room temperature for 1 h. The immunostaining was analyzed by using a laser scanning confocal microscope (PCM 200, Nikon).

The paraffin-embedded thin (6-μm-thick) human brain tissue sections were first deparaffinized, oxidized, and washed. The nonspecific binding of the sections was blocked with 5% normal goat serum for 30-45 min, followed by incubation with the monoclonal antibody RL2 (1:200) at 4° C. overnight and then with biotinylated anti-mouse lgG. The immunostaining was developed by using avidin/biotinylated horseradish peroxidase (Santa Cruz Biotechnology) and peroxidase substrate DAB. The stained sections were finally mounted on microscope slides (Brain Research Laboratories, Newton, Mass.), dehydrated, and covered with coverslips.

### Statistical Analysis

The data were analyzed by one-way ANOVA followed by Tukey's post hoc tests or unpaired two-tailed t test using software Graphpad Prism 5. All data are presented as means±SEM, and p<0.05 was considered statistically significant.

What is claimed is:

1. A method of treating a patient for ischemia-reperfusion-induced brain injury, comprising the step of modulating the levels of O-GlcNAcylation in the brain of the patient.

2. The method of claim 1, wherein the step of modulating the levels of O-GlcNAcylation in the brain of the patient comprises administering a therapeutic amount of a compound that increases the hexosamine biosynthesis pathway flux that bypasses glutamine/fructose-6-phosphate amidotransferase 2.

3. The method of claim 2, wherein the compound comprises glucosamine.

4. The method of claim 3, wherein the glucosomine is administered by intravenous injection.

5. The method of claim 4, wherein the dosage is between 1 and 10 mg per day.

6. The method of claim 5, wherein the treatment begins within three hours of the ischemia-reperfusion-induced brain injury and continues for at least two days.

7. The method of claim 1, wherein the step of modulating the levels of O-GlcNAcylation in the brain of the patient comprises administering a therapeutic amount of a compound that inhibits OGA.

8. The method of claim 7, wherein the compound comprises thiamet-G.

9. The method of claim 8, wherein the thiamet-G is administered by intravenous injection.

10. The method of claim 9, wherein the dosage is between 0.1 and 1 mg per day.

11. The method of claim 8, wherein the thiamet-G is administered orally.

12. The method of claim 11, wherein the dosage is between 1 and 10 mg per day.

13. The method of claim 7, wherein the compound comprises GlcNAcstatin G.

14. The method of claim 13, wherein the GlcNAcstatin G is administered by intravenous injection.

15. The method of claim 9, wherein the dosage is between 0.02 and 0.2 mcg per day.

16. The method of claim 13, wherein the GlcNAcstatin G is administered orally.

17. The method of claim 16, wherein the dosage is between 0.2 and 2 mcg per day.
